(19) United States
(12) Patent Application Publication
(10) Pub. No.: US 2025/0239325 A1
(43) Pub. Date: Jul. 24, 2025

(54) BINDING AFFINITY PREDICTION USING 3D GNNS

(71) Applicant: NEC Laboratories America, Inc., Princeton, NJ (US)

(72) Inventors: Jonathan Warrell, Princeton, NJ (US); Renqiang Min, Princeton, NJ (US); Yueyu Jiang, Highland Park, NJ (US)

(21) Appl. No.: 19/024,350

(22) Filed: Jan. 16, 2025

Related U.S. Application Data
(60) Provisional application No. 63/622,166, filed on Jan. 18, 2024.
(60) Provisional application No. 63/687,397, filed on Aug. 27, 2024.

(51) Int. Cl. G16B 15/30; G06F 30/27; G16B 15/20; G16B 40/20

(52) U.S. Cl. CPC G16B 15/30; G06F 30/27; G16B 15/20; G16B 40/20

(57) ABSTRACT

Methods and systems for peptide binding prediction include predicting a three-dimensional (3D) structure of a peptide and a major histocompatibility (MHC) complex to generate a graph. The 3D structure is refined by pruning edges of the graph having a distance between the peptide and the MHC complex that is below a threshold value. Models for MHC-I and MHC-II binding prediction are trained, including Bayesian reweighting of data for the MHC-II binding prediction, using the pruned graph.

This application claims priority to U.S. Patent Application No. 63/622,166, filed on Jan. 18, 2024, and to U.S. Patent Application No. 63/687,397, filed on Aug. 27, 2024, each incorporated herein by reference in its entirety.

### BACKGROUND

### Technical Field

The present invention relates to protein binding prediction and, more particularly, to graph neural network (GNN) models that predict protein binding.

### Description of the Related Art

Immunotherapy aims at boosting a patient's immune system against pathogens and tumor cells. The immune response is triggered when immune cells recognize foreign peptides, presented by major histocompatibility complex (MHC) proteins on a cell's surface. To be recognized, the foreign peptides are bound to MHC Class I and Class II proteins. The resulting peptide-MHC complexes interact with T cell receptors. These interactions can be leveraged to generate peptide-based vaccines to prevent disease.

### SUMMARY

A method for peptide binding prediction includes predicting a three-dimensional (3D) structure of a peptide and a major histocompatibility (MHC) complex to generate a graph. The 3D structure is refined by pruning edges of the graph having a distance between the peptide and the MHC complex that is below a threshold value. Models for MHC-I and MHC-II binding prediction are trained, including Bayesian reweighting of data for the MHC-II binding prediction, using the pruned graph.

A system for peptide binding prediction includes a hardware processor and a memory that stores a computer program. When executed by the hardware processor, the computer program causes the hardware processor to predict a 3D structure of a peptide and a MHC complex to generate a graph, to refine the 3D structure by pruning edges of the graph having a distance between the peptide and the MHC complex that is below a threshold value, and to train models for MHC-I and MHC-II binding prediction, including Bayesian reweighting of data for the MHC-II binding prediction, using the pruned graph.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram illustrating binding between a peptide and a major histocompatibility (MHC) complex, in accordance with an embodiment of the present invention;

FIG. 2 is a block/flow diagram of a method of training a binding prediction model, in accordance with an embodiment of the present invention;

FIG. 3 is a block/flow diagram of a method for developing and administering a new treatment using peptide binding prediction, in accordance with an embodiment of the present invention;

FIG. 4 is a block diagram of a healthcare facility that uses peptide binding prediction to generate and administer treatments to patients, in accordance with an embodiment of the present invention;

FIG. 5 is a block diagram of a computing device that can train a peptide binding prediction model and administer treatment, in accordance with an embodiment of the present invention;

FIG. 6 is a diagram of an exemplary neural network architecture that can be used to implement part of a peptide binding prediction model, in accordance with an embodiment of the present invention; and

FIG. 7 is a diagram of an exemplary deep neural network architecture that can be used to implement part of a peptide binding prediction model, in accordance with an embodiment of the present invention.

### DETAILED DESCRIPTION OF PREFERRED EMBODIMENTS

Testing a given peptide for its binding affinity to a major histocompatibility complex (MHC) is a time-consuming process, but is needed when developing new immunotherapies. The binding affinity for a peptide can be predicted using machine learning techniques, so that large numbers of peptides and MHC molecules may be quickly screened for binding affinity.

However, there may be a large amount of variation in MHCs and peptide sequences. In particular, MHC-II proteins exhibit larger variation than MHC-I proteins and bind to peptide sequences of longer lengths (e.g., up to 12 amino acids). Machine learning systems may therefore be used to generalize to MHC alleles and peptides that are not seen during training, with three-dimensional (3D) structure prediction being used to help with the sparsity of experimental structural data available for most alleles.

A 3D structure for an MHC-peptide complex is predicted and then a graph neural network (GNN) may be used to predict the binding affinity of the complex. Graph structure learning may further be used to refine a partial graph prior to prediction, to help address potential inaccuracies in the predicted 3D structures. This graph structure learning may employ a variational optimization to train meta-features for the graph refinement. Data reweighting may then be used to maximize the use of information shared between MHC-I and MHC-II proteins, reweighting class-I molecules when training a class-II predictor (and vice-versa) to provide information sharing between the classes.

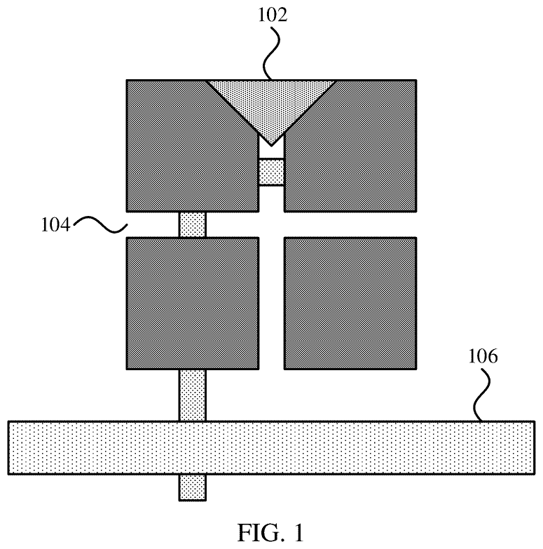

Referring now to FIG. 1, a diagram of a peptide-MHC protein bond is shown. A peptide 102 is shown as binding with an MHC protein 104, with complementary two-dimensional interfaces of the figure suggesting complementary shapes of these three-dimensional structures. The MHC protein 104 may be attached to a cell surface 106.

An MHC is an area on a DNA strand that codes for cell surface proteins that are used by the immune system. MHC molecules are used by the immune system and contribute to the interactions of white blood cells with other cells. For example, MHC proteins impact organ compatibility when performing transplants and are also important to vaccine creation.

A peptide, meanwhile, may be a portion of a protein. When a pathogen presents peptides that are recognized by a MHC protein, the immune system triggers a response to destroy the pathogen. Thus, by finding peptide structures that bind with MHC proteins, an immune response may be intentionally triggered, without introducing the pathogen itself to a body. When evaluating a peptide for its ability to bind to the MHC protein 104, a score may be generated that reflects the binding affinity between the two.

Interactions between peptides and MHCs play a role in cell-mediated immunity, regulation of immune responses, and transplant rejection. Prediction of peptide-protein binding helps guide the search for, and design of, peptides that may be used in vaccines and other medicines.

Thus, given a particular genome (e.g., sequenced from a tumor cell), peptide sequences may be extracted to generate a library of peptides that uniquely identifies the pathogen. By targeting this library, peptides can be screened/selected that bind to MHCs that are present on cell surfaces, so that immune responses can be triggered to kill the pathogen or tumor cells.

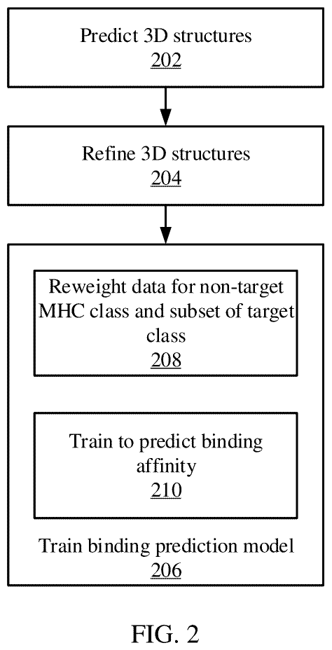

Referring now to FIG. 2, a method of training a binding prediction model is shown. Training data may be provided in the form of sequence pairs, represented as (sᵢ^{M}, sᵢ^{P}), with sᵢ^{M} representing matched MHC and with sᵢ^{P} representing matched peptide amino acid sequences for training instance i. Binary labels yᵢ∈{0,1} indicate whether a given instance is a binding or non-binding pair. The 3D structure of each training instance may be predicted in block 202, for example using a deep learning protein folding system. The structure may be initialized using template alignment, followed by refinement of the spatial coordinates using pretrained model parameters in block 204. A distance threshold τ_{d} is used to generate a 3D graph associated with each training instance, Gᵢ=(Xᵢ, Eᵢ), where the node features Xᵢ are formed by concatenating a 1-hot encoding of the amino acid, five physico-chemical features, and a binary indicator for whether the residue is part of the MHC (0) or peptide (1) chain, and edges are placed between each pair of amino acids whose central carbon atom lies within τ_{d} of each other. The node features Xᵢ thus have dimensionality Nᵢ×D_{X}, where Nᵢ is the number of nodes in graph i and D_{X}=28.

The edges connecting the MHC and peptide chains may be particularly important in predicting the binding affinity of a given pair. The model may therefore learn an additional set of ‘meta-features,’ which may be used to prune the edges between these chains in an instance-specific fashion. Hence, a matrix of latent meta-features, Zᵢ, may be predicted for each instance i, with dimensionality Nᵢ×D_{z}, where D_{z} is the metafeature dimensionality. The latent meta-features are learned as a linear transformation of the image features, i.e. Zᵢ=XᵢW₀. Using the trained meta-features, the refined graph Eᵢ′ may be formed by pruning the edges between the chains whose distance is below a pre-defined threshold ϵ:

$${E}_{i}^{′} ( {n}_{1} , {n}_{2} ) = \{ \begin{matrix}{E}_{i}^{′} ( {n}_{1} , {n}_{2} ) & if C ( {n}_{1} ) = C ( {n}_{2} ) \\ {‖ {z}_{i}^{{n}_{1}} - {z}_{i}^{{n}_{2}} ‖}_{2} \leq ϵ & otherwise\end{matrix}$$

where n₁, n₂∈{1 . . . Nᵢ}, ∥⋅∥₂ is the Euclidean norm, and C(nⱼ) is a binary indicator for whether node j is part of the MHC(0) or peptide (1) chain.

The binding affinity label of a given pair is predicted by performing message passing on the refined graphs, Gᵢ′=(Xᵢ, Eᵢ′). The network is parameterized by weight matrices W_{1 . . . L}, where L is the number of layers in the GNN, with Wₗ having dimensionality Dₗ₋₁×Dₗ, such that Dₗ is the number of hidden units per node in layer l, and D₀=D_{X}, D_{L}=1. The values of {L, D_{1 . . . L-1}} are treated as additional hyperparameters. The message-passing updates can be written as:

$${x}_{n}^{l} = σ ( {\sum }_{\{ m \vert ( m , n ) ∈ {E}^{′} \}} \frac{{x}_{m}^{l - 1} {W}_{l}}{\sqrt{deg ( n ) deg ( m )}} )$$

for levels l<L, where σ(x)=max(0, x) is the rectified linear unit (ReLU) function, and deg(n) is the degree of node n. For level L, mean-pooling followed by a final fully-connected linear is used to generate the log-odds of the predicted output; hence: x^{L}=meanₙ(xₙ^{L-1})W_{L}. Training 206 for an adaptive 3D-GNN is performed using a training loss:

$$L ( 𝒳 , \vert {W}_{0 … L} ) = {\sum }_{i} {L}_{i} ( {G}_{i} , {Y}_{i} \vert {W}_{0 … L} ) , {L}_{i} ( {G}_{i} , {Y}_{i} \vert {W}_{0 … L} ) = CrossEntropy ( {Y}_{i} , \frac{1}{( 1 - exp ( {x}_{i}^{L} ) )} ) ,$$

where CrossEntropy (y, p)=−y log(p)−(1−y)log(1−p), [symbol] ={(sᵢ^{M}, sᵢ^{P})|i=1 . . . N}, and [symbol] ={yᵢ|i=1 . . . N} with yᵢ being graph labels.

The model may be fine-tuned to optimize performance on MHC-I and MHC-II binding prediction separately. Since the diversity of training instances for MHC-II complexes is high, a Bayesian data reweighting scheme is used to reweight data for MHC classes 208 when training the MHC-II specific model. Such an approach may not be needed for the MHC-I predictor.

A reweighted likelihood may therefore be used:

$$P ( 𝒳 , , ω \vert {W}_{0 … L} ) = \frac{1}{Z} {\prod }_{i = 1} {{P}_{{ω}_{i}} ( {ω}_{i} )}^{{λ}_{2}} {P ( {G}_{i} , {Y}_{i} \vert {W}_{0} … L )}^{{λ}_{1} {ω}_{i}}$$

where ωᵢ∈[0 1] is a weight associated with data-point i, P_{ω} is a prior on the model data-instance weights, λ₁ and λ₂ are hyper-parameters, and Z is a normalizing constant. For each training run, a subset of the training data is held in reserve as a validation set. Hence, when training the MHC-II model, P_{ω}_{ᵢ}=δ(.; 1) for the validation instances, where δ(.|a) is a delta distribution centered at a, while for the remaining data-points, P_{w}_{ᵢ}= [symbol] (.; 1,1), where [symbol] (.; a, b) is a normal distribution with mean a and standard deviation b. The following loss may be used when training the reweighted form of the model to predict MHC-II binding:

$${L}_{RW} ( 𝒳 , \vert {W}_{0 … L} , ω ) = {\sum }_{i ∈ {S}_{2}} {L}_{i} ( {G}_{i} , {Y}_{i} \vert {W}_{0 … L} ) + {λ}_{1} {\sum }_{i ∈ {S}_{1}} {ω}_{i} {L}_{i} ( {G}_{i} , {Y}_{i} \vert {W}_{0 … L} ) + {λ}_{2} {\sum }_{i ∈ {S}_{2}} {( 1 - {ω}_{i} )}^{2}$$

where S₁ and S₂ are the sets of data instances associated with training and validation sequences respectively, and Lᵢ is defined as in Eq. 4. To enforce the constraint ωᵢ∈[0 1] for i∈S₁, auxiliary variables ωᵢ′∈ [symbol] may be used for these data-points, with ωᵢ=1/(1+exp(−ωᵢ′)) while optimizing over ωᵢ′ and W₀ . . . L. Since Z is a global normalizing factor in P( [symbol] , [symbol] , ω|W_{0 . . . L}), optimizing L_{RW}( [symbol] , [symbol] |W_{0 . . . L}, ω) is equivalent to performing maximum a posteriori (MAP) inference on (W, ω). Block 210 trains the GNN using the 3D structure and an amino-acid physical features model to predict binding affinity, using the reweighted data.

In training the model to predict binding to MHC Class II molecules, all MHC-I-peptide pairs may be included in S₁ and a subset of MHC-II-peptide pairs (e.g., about 80%), while the remaining MHC-II-peptide pairs (20%) may be included in S₂. This allows the model to select which MHC-I-peptide pairs are relevant to the MHC-II predictive task during training by reweighting these examples in the loss function according to how much they help reduce the loss over S₂. The reweighted loss above is an expansion of the log of the reweighted likelihood stated previously for the case just specified; the factor (1/Z) is an additive constant in the loss, and hence may be ignored, while the second term in the reweighted likelihood becomes the first two terms in the loss, where it is split across the S₁ and S₂ instances. To train the model to predict MHC-I binding, an unweighted loss L( [symbol] , [symbol] |W_{0 . . . L}) may be used as described above, or the contents of S₁ and S₂ may be changed above to contain only MHC-II-peptide pairs, and mixed MHC-I and MHC-II-peptide pairs respectively.

Since the projection W₀ determines the meta-feature matrix Z, which in turn determines the graph structure of the adapted (refined) spatial graph G′ used for message passing, there is a complex interaction between a discrete optimization over the space of refined spatial graphs (implicitly parameterized by W₀) and the continuous predictions of the network, determined by W_{1 . . . L}. The underlying objective is therefore discontinuous at points where changing W₀ changes the refined graphs. However, if W₀ is held constant, the objective is continuous over the remaining parameters, and can be handled by gradient descent.

A modified form of variational optimization (VO) is used, which makes it possible to convert an objective with discontinuities into a continuous objective. This is done by introducing a variational distribution Q over the parameters W₀, which may be a Gaussian with a symmetric covariance matrix. At a given meta-epoch t, this variational distribution has the form:

$$vec ( {W}_{0}^{t} ) \sim ( . \vert {μ}_{t} , {σ}_{t} I )$$

where vec(W₀) is the vectorization of matrix W₀, μₜ is a vector of mean values, σₜ is a scalar, and I is the identity matrix. At meta-epoch t, S samples are drawn from vec(W₀^{t}), W₀¹ . . . W₀^{S}. For the raw cross-entropy loss (i.e. without data reweighting), the parameters W_{1 . . . L} may be optimized using gradient descent, to find W_{1 . . . L}^{s} for s=1 . . . S (where a portion of the training data is reserved as a validation set to perform early stopping). Hence, the sample training loss at meta-epoch t is calculated as:

$${L}_{s}^{t} = L ( 𝒳 , \vert {W}_{0 … L}^{ts} )$$

where L is the cross-entropy loss. Alternatively, in the case that Bayesian data reweighting is applied during training, both W_{1 . . . L}^{s} and ωₛ are optimized for s=1 . . . S using gradient descent while fixing W₀^{g}, and let Lₛ^{t}=L_{RW}( [symbol] , [symbol] |W^{ts}, ω^{ts}) using L_{RW}.

Smoothing-based optimization (SMO) is used to update μ and σ:

$${μ}_{t + 1} = \frac{{\sum }_{s} {F}_{s} vec ( {W}_{0}^{t} )}{{\sum }_{s} {F}_{s}}$$

$${σ}_{t + 1} = \sqrt{\frac{{\sum }_{s} {F}_{s} {\vert vec ( {W}_{0}^{t} ) - {μ}_{t} \vert}_{2}^{2}}{{D}_{X} {D}_{Z} {\sum }_{s} {F}_{s}}}$$

where Fₛ=max(−Lₛ+c, 0) is the score for sample s, with the offset c>0 set to a positive constant. The value of c is treated as an additional hyperparameter, which is set empirically to ensure that −Lₛ+c>0 for observed values of Lₛ. These updates improve the value of F (i.e. the inverse loss) in expectation; hence:

$${𝔼}_{{W}_{0} \sim {Q}_{t + 1}} [ F ( 𝒳 , \vert {W}_{0} … L ) ] \geq {𝔼}_{{W}_{0} \sim {Q}_{t}} [ F ( 𝒳 , \vert {W}_{0 … L} ) ]$$

where Qₜ= [symbol] (.|μₜ, σₜ) is the variational distribution over W₀ at meta-epoch t, and [symbol] [.] is the expectation operator.

By optimizing [symbol] _{W}_{₀}_{˜Q}[F( [symbol] , [symbol] |W_{0 . . . L})], a lower bound on the whole objective function L or L_{RW} is optimized, as

$${𝔼}_{Q} [ F ( θ ) ] \leq {max}_{θ} F ( θ ) ,$$

where θ={W_{0 . . . L}}. As F is continuous within local regions the smoothing objective is strengthened by using gradient descent above, F(θ)≥F(θ₀), where θ₀ is the initialization of W_{0 . . . L} when using gradient descent. The approach above thus relies on the following variational bound, where the performance of the parameters using gradient descent are sandwiched between the initial parameters and the optimal performance:

$${𝔼}_{Q} [ F ( {θ}_{0} ) ] \leq {𝔼}_{Q} [ F ( θ ) ] \leq {max}_{θ} F ( θ )$$

After training, a set of parameters is produced for an adaptive GNN model, which is optimized to predict the binding affinity of MHC-I or MHC-II complexes with novel peptides. Given a novel peptide target then, for instance from a virus or a patient's cancer, the expected immunogenicity (the degree to which the peptide will be presented to the immune system) of this peptide for a given patient may be predicted by running the model on the peptide in combination with the repertoire of MHC-I and MHC-II molecules harbored by the patient. This approach may be used to filter potential target peptides, prior to designing a TCR antibody to target a specific MHC-peptide complex.

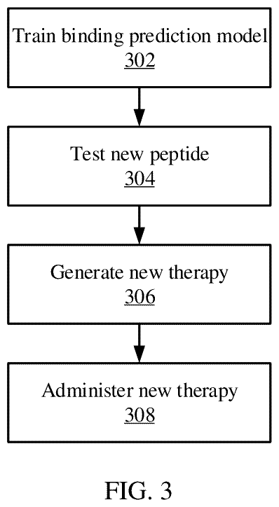

Referring now to FIG. 3, a method of creating and administering a new therapy is shown. Block 302 trains a binding prediction model, as described above. Block 304 then tests a new peptide for binding with a particular MHC complex. The peptide may be derived from a particular pathogen or cancer, so that binding to the MHC complex may trigger an appropriate immune response.

Based on the outcome of block 304, block 306 generates a new therapy for the pathogen or cancer. For example, the trained model may output a score which indicates a strong binding affinity between the new peptide and the MHC complex. This peptide may be used to trigger an immune response that targets the pathogen or cancer. Block 308 then administers the new therapy to a patient.

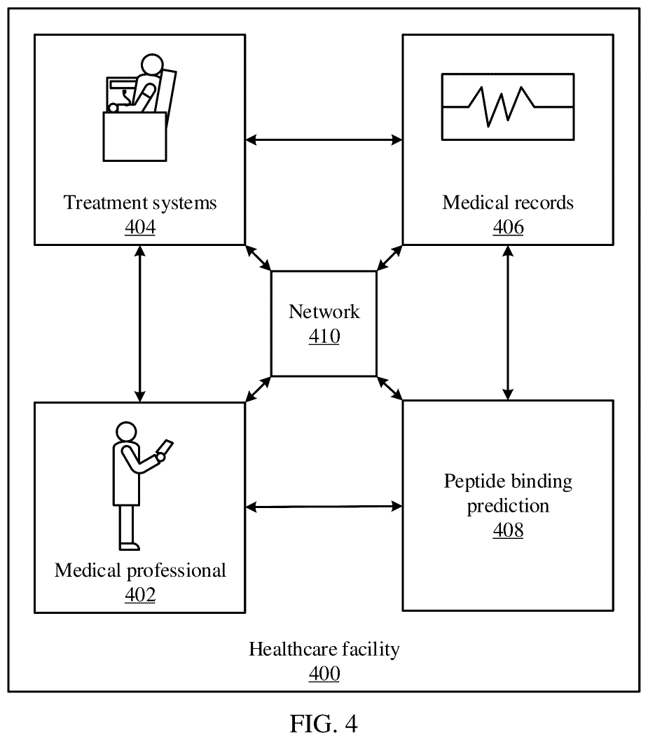

Referring now to FIG. 4, a diagram of information extraction is shown in the context of a healthcare facility 400. Peptide binding prediction 408 may be used to identify peptides that bind with MHC complexes, to aid with medical decision making and treatment. The peptide binding prediction 408 may be used to generate treatment recommendations relating to a patient's medical condition based on up-to-date medical records 406.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Based on information provided by the peptide binding prediction 408, the medical professionals 402 may make medical decisions about patient healthcare suited to the patient's needs. For example, the medical professionals 402 may make a diagnosis of the patient's health condition and may prescribe particular medications, surgeries, and/or therapies.

The different elements of the healthcare facility 400 may communicate with one another via a network 410, for example using any appropriate wired or wireless communications protocol and medium. Thus the peptide binding prediction 408 can be used to design a treatment that targets a patient's specific condition, for example using tissue samples and medical records 406. The treatment systems 404 may be used to generate and administer a therapy based on a peptide binding prediction 408.

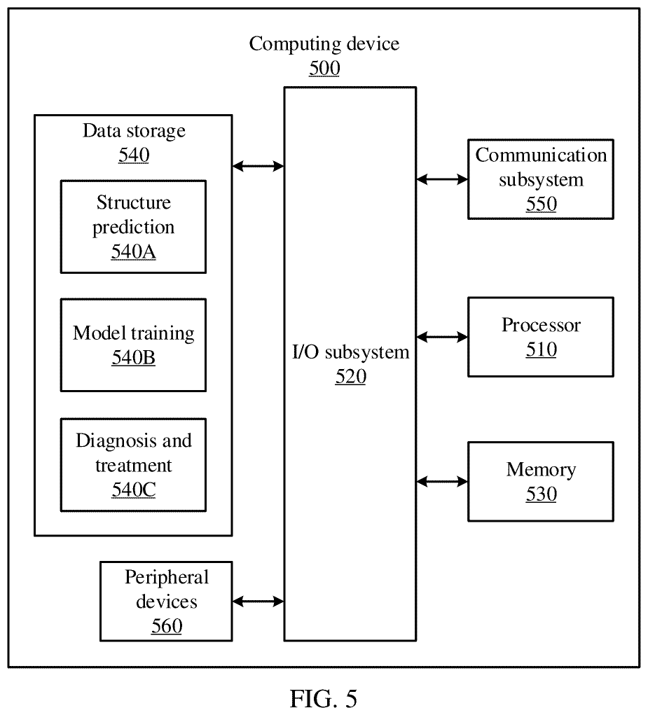

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for predicting molecule structure, 540B for training a peptide binding prediction model, and/or 540C for generating a treatment based on a patient's condition. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

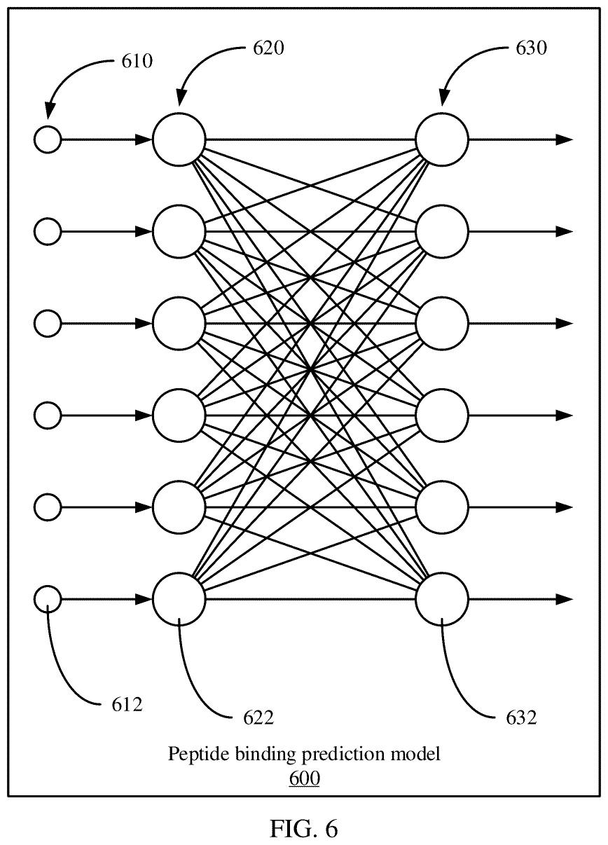

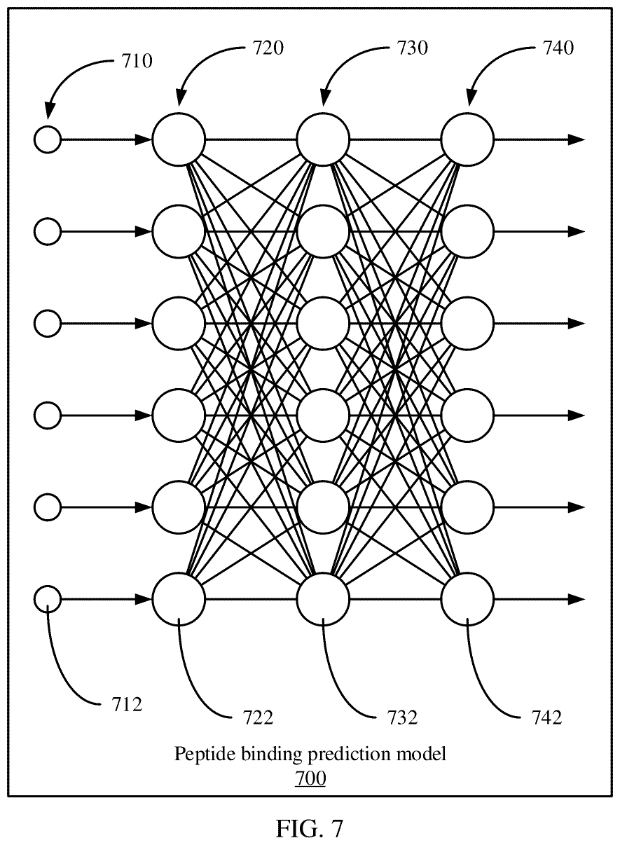

Referring now to FIGS. 6 and 7, exemplary neural network architectures are shown, which may be used to implement parts of the present models, such as the classifier 122. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for peptide binding prediction, comprising:
predicting a three-dimensional (3D) structure of a peptide and a major histocompatibility (MHC) complex to generate a graph;
refining the 3D structure by pruning edges of the graph having a distance between the peptide and the MHC complex that is below a threshold value; and
training models for MHC-I and MHC-II binding prediction, including Bayesian reweighting of data for the MHC-II binding prediction, using the pruned graph.

2. The method of claim 1, wherein the models for MHC-I and MHC-II binding prediction are respective graph neural network machine learning models that output a binding affinity label for an input peptide.

3. The method of claim 1, wherein training the models for MHC-I and MHC-II binding prediction uses different loss functions for each.

4. The method of claim 3, wherein training the model for MHC-I binding prediction uses a cross-entropy loss.

5. The method of claim 3, wherein training the model for MHC-II binding prediction uses a loss function that has separately weighted cross-entropy loss parts for a training dataset and a validation dataset.

6. The method of claim 1, wherein training the models includes variational optimization with smoothing-based optimization based on either a training likelihood or reweighted likelihood.

7. The method of claim 1, wherein training the model includes a reweighting loss term:
$${L}_{RW} = {\sum }_{i ∈ {S}_{2}} {L}_{i} ( {G}_{i} , {Y}_{i} \vert {W}_{0 … L} ) + {λ}_{1} {\sum }_{i ∈ {S}_{1}} {ω}_{i} {L}_{i} ( {G}_{i} , {Y}_{i} \vert {W}_{0 … L} ) + {λ}_{2} {\sum }_{i ∈ {S}_{2}} {( 1 - {ω}_{i} )}^{2}$$
where Gᵢ are graphs, Yᵢ are labels, W_{0 . . . L} are weight matrices, ωᵢ are weights associated with data-point i, λ₁ and λ₂ are hyper-parameters, S₁ and S₂ are sets of data instances associated with training and validation sequences respectively, and Lᵢ is a cross-entropy loss.

8. The method of claim 1, further comprising generating a binding prediction using the trained models based on an input peptide for medical decision making.

9. The method of claim 8, further comprising administering a treatment to a patient based on the binding prediction, wherein the input peptide is derived from a sample taken from the patient.

10. The method of claim 7, wherein generating the binding prediction includes message passing on pruned graph.

11. A system for peptide binding prediction, comprising:
a hardware processor; and
a memory that stores a computer program that, when executed by the hardware processor, causes the hardware processor to:
predict a three-dimensional (3D) structure of a peptide and a major histocompatibility (MHC) complex to generate a graph;
refine the 3D structure by pruning edges of the graph having a distance between the peptide and the MHC complex that is below a threshold value; and
train models for MHC-I and MHC-II binding prediction, including Bayesian reweighting of data for the MHC-II binding prediction, using the pruned graph.

12. The system of claim 11, wherein the models for MHC-I and MHC-II binding prediction are respective graph neural network machine learning models that output a binding affinity label for an input peptide.

13. The system of claim 11, wherein the computer program further causes the hardware processor to train the models for MHC-I and MHC-II binding prediction using different loss functions for each.

14. The system of claim 13, wherein the computer program further causes the hardware processor to use a cross-entropy loss to train the model for MHC-I binding prediction.

15. The system of claim 13, wherein the computer program further causes the hardware processor to use a loss function that has separately weighted cross-entropy loss parts for a training dataset and a validation dataset to train the model for MHC-II binding prediction.

16. The system of claim 11, wherein the computer program further causes the hardware processor to perform variational optimization with smoothing-based optimization to train the models applied to a loss function based on either a training likelihood or reweighted likelihood.

17. The system of claim 11, wherein training the model includes a reweighting loss term:
$${L}_{RW} = {\sum }_{i ∈ {S}_{2}} {L}_{i} ( {G}_{i} , {Y}_{i} \vert {W}_{0 … L} ) + {λ}_{1} {\sum }_{i ∈ {S}_{1}} {ω}_{i} {L}_{i} ( {G}_{i} , {Y}_{i} \vert {W}_{0 … L} ) + {λ}_{2} {\sum }_{i ∈ {S}_{2}} {( 1 - {ω}_{i} )}^{2}$$
where Gᵢ are graphs, Yᵢ are labels, W_{0 . . . L} are weight matrices, ωᵢ are weights associated with data-point i, λ₁ and λ₂ are hyper-parameters, S₁ and S₂ are sets of data instances associated with training and validation sequences respectively, and Lᵢ is a cross-entropy loss.

18. The system of claim 11, wherein the computer program further causes the hardware processor generate a binding prediction using the trained models based on an input peptide for medical decision making.

19. The system of claim 18, wherein the computer program further causes the hardware processor administer a treatment to a patient based on the binding prediction, wherein the input peptide is derived from a sample taken from the patient.

20. The system of claim 17, wherein generation of the binding prediction includes message passing on pruned graph.
